(19) United States
(12) Patent Application Publication
(10) Pub. No.: US 2015/0118267 A1
(43) Pub. Date: Apr. 30, 2015

(54) METHOD OF USING THEAFLAVIN

(71) Applicants: Shiming Li, Glastonbury, CT (US); Xueyin Ren, Jiangyin City (CN); Jianhong Liu, Jiangyin City (CN)

(72) Inventors: Shiming Li, Glastonbury, CT (US); Xueyin Ren, Jiangyin City (CN); Jianhong Liu, Jiangyin City (CN)

(73) Assignee: Dongning Li, Jiangyin City (CN)

(21) Appl. No.: 14/527,610

(22) Filed: Oct. 29, 2014

Related U.S. Application Data
(60) Provisional application No. 61/897,812, filed on Oct. 30, 2013.

(51) Int. Cl. A61K 31/353; A61K 36/82

(52) U.S. Cl. CPC A61K 31/353; A61K 36/82
USPC 424/278.1; 514/456; 424/729

(57) ABSTRACT

A method for enhancing the immune system or for treating an infectious disease, the method includes administering to a subject in need thereof an effective amount of theaflavins, analogs thereof, or prodrugs thereof, or includes administering to a subject a composition comprising a physiologically acceptable carrier and an effective amount of an extract of tea.

### CROSS REFERENCE TO RELATED APPLICATIONS

This application claims the benefit of U.S. Provisional Application No. 61/897,812 filed on Oct. 30, 2013. The contents of both applications are hereby incorporated by reference in their entirety.

### FIELD OF THE INVENTION

This disclosure relates to the field of enhancing the immune system or treating an infectious disease and use of theaflavins, analogs thereof, or prodrugs thereof to enhance the immune system or treat an infectious disease, and use of a composition having a theaflavin-containing tea extract to perform the same.

### BACKGROUND OF THE INVENTION

The function of the immune system is to defend the body against invaders like viruses, fungi, cancer cells and other substances foreign to the body as the body's is immune system attacks and eliminates bacteria and other foreign substances as well as cancer cells. When the immune system is weakened, diseases such as cancer or those caused by infectious pathogens occur more likely. For example, the immune system's important role in controlling cancer cell development is shown by such statistic: Cancer is 100 times more likely to occur in people who take drugs that suppress the immune system in cases of an organ transplant or a rheumatic disease than in people with normal immune systems.

An infectious disease refers to illness resulting from being infected after a contact with pathogens that include viruses, bacteria, fungi, protozoa, multicellular parasites. The body's immune system plays a critical role in defending against an infectious disease, a process requires immune mechanisms to kill or inactivate the inoculum of the pathogen. A specifically acquired immunity against an infectious disease may be mediated by antibodies and/or T lymphocytes. As such, to promote the effectiveness or efficiency of the immune system becomes one of the most effective approaches to prevent or treat an infectious disease.

There is a need to enhance the immune system or boost immune responses. There also remains a need for an effective prevention and treatment of infectious diseases without severe side effects through, as set forth above, promoting the effectiveness or efficiency of the immune system.

### SUMMARY OF THE INVENTION

This invention provides a method of using theaflavins or an theaflavin-containing tea extract, which, unexpectedly, show an advantage in enhancing the io immune system. As such, it can be used for treating an infectious disease.

One aspect of this invention relates to a method that includes administering to a subject in need thereof an effective amount of theaflavins, analogs thereof, or prodrugs thereof.

The theaflavins includes theaflavin, theaflavin-3-gallate, theaflavin-3′-gallate, is theaflavin 3,3′-digallate, or a combination thereof.

The theaflavins, analogs thereof, or prodrugs thereof, in a solid, semi-solid, liquid, or gaseous form, can be administered to the subject in a dosage equivalent to 5-500 mg (preferably, 10-100 mg) of theaflavin per kg of body weight per day. This administration can be performed orally, buccally, rectally, parenterally, intravenously, intraperitoneally, intradermal, subcutaneously, intramuscularly, trans-dermally, or intratracheally to the subject.

Examples of the alcoholic liver disease include colds, flu, leprosy, skin infections, acne, a urinary tract infection, and a respiratory infection.

The method of this invention can further include administering to the subject a second therapeutic agent.

Another aspect of this invention relates to a method for enhancing the immune system or improving the immune response sensitivity of the immune system that includes administering to a subject in need thereof a composition comprising a physiologically acceptable carrier and an effective amount of an extract of tea. This method can further include administering to the subject a second therapeutic agent.

The extract of tea contains theaflavin, theaflavin-3-gallate, theaflavin-3′-gallate, theaflavin 3,3′-digallate, or a combination thereof. It can also contain catechins, (+)-catechin, (−)-epicatechin, (−)-epigallocatechin, (−)-epicatechin gallate, (−)-epigallocatechin gallate, gallocatechin, catechin gallate, gallocatechin gallate, or a combination thereof.

The extract of tea can be administered to the subject in an amount equivalent to 5-500 mg (preferably, 10-100 mg) of theaflavin per kg of body weight per day. The composition is a pharmaceutical composition that can be in a solid, semi-solid, liquid, or gaseous form.

Yet another aspect of this invention relates to a method for treating an infectious disease. The method includes administering to a subject in need thereof a composition comprising a physiologically acceptable carrier and an effective amount is of an extract of tea.

Ingredients and dosages of the extract of tea are enumerated above. Examples of the composition and the infectious disease are also enumerated above.

The details of the invention are set forth in the description below. Other features, objects, and advantages of the invention will be apparent from the description and from the claims.

### DETAILED DESCRIPTION

The present invention provides a method for enhancing the immune system or for treating an infectious disease that includes administering to a subject in need thereof an effective amount of theaflavins, analogs thereof, or prodrugs thereof.

Theaflavins are a class of benzoannulenone compounds which are formed from oxidation reactions of polyphenolic compounds. There are 12 components in theaflavins, including theaflavin (TF), theaflavin-3-gallate (TFMG), theaflavin-3,3′-digallate (TFdiG) and theaflavin-3′-gallate (TFM′G), which are the four major components. Pure theaflavins are orange colors, form needle crystals, have melting points of 237-240° C., are soluble in water, methanol, ethanol, acetone, n-butanol and ethyl acetate, are slightly soluble in ethyl ether, and are insoluble in chloroform and benzene. Theaflavins in solution are clear orange in color and are slightly acidic with a pH value of about 5.7. The solution color is not affected by the pH of the tea extraction solution, but theaflavins are auto-oxidative in basic solution. The oxidation process increases with the pH value.

Thearubigins are a class of complex, inhomogeneous brown colored phenolic compounds, with a range of molecular weight of 1,000-40×10³. Due to inhomogeneity, unclear structure, and unknown properties, it is difficult to isolate and purify the thearubigins.

Tea polyphenols, including catechins and theaflavins, are known for reducing triglyceride, removing free radicals, having anti-oxidant, anti-bacteria, anti-virus, anti-tumor, anti-mutagenic, and odor removal properties, and treating cardiovascular is diseases, etc. They are applied in pharmaceutical, nutraceutical and food additive fields.

One example of the method is that administration of theaflavins, analogs thereof, or prodrugs thereof can be in monotherapy or in combination therapy. The combination therapy comprises administering to a subject in need thereof an effective amount of the theaflavins, analogs thereof, or prodrugs thereof in combination with an effective amount of a second therapeutic agent.

Another aspect of the invention is a method of enhancing the immune system or improving the immune response sensitivity of the immune system that comprises administering to a subject in need thereof a composition comprising a physiologically acceptable carrier and an effective amount of an extract of tea.

The extract of tea includes theaflavin, theaflavin-3-gallate, theaflavin-3′-gallate, theaflavin 3,3′-digallate, or a combination thereof.

Preferably, an extract of tea further includes catechins, (+)-catechin, (−)-epicatechin, (−)-epigallocatechin, (−)-epicatechin gallate, (−)-epigallocatechin gallate, gallocatechin, catechin gallate, gallocatechin gallate or a combination thereof.

An example of the method is wherein administration of the composition can be by orally, buccally, rectally, parenterally, intravenously, intraperitoneally, intradermal, subcutaneously, intramuscularly, trans-dermally, or intratracheally.

An example of the method is wherein theaflavin is administered in an amount equivalent of theaflavin from about 5 mg to about 500 mg per kg of body weight.

Yet another aspect of the invention is a method of improving the immune response sensitivity of the immune system that comprises administering to a subject in need thereof a composition comprising a physiologically acceptable carrier and an effective amount of an extract of tea.

Also within the scope of this invention is a method for treating an infectious disease that comprises administering to a subject in need thereof an effective amount of theaflavin or a theaflavin analog. is The term infectious disease includes colds, flu, leprosy, skin infections, acne, urinary tract infections, respiratory infections, and others.

One example of the method is that administration of theaflavin or a theaflavin analog can be in monotherapy or in combination therapy. The combination therapy comprises administering to a subject in need thereof an effective amount of theaflavin or a theaflavin analog in combination with an effective amount of a second therapeutic agent.

Another aspect of the invention is a method for treating an infectious disease that comprises administering to a subject in need thereof a composition comprising a physiologically acceptable carrier and an effective amount of an extract of tea.

An example of the method is wherein administration of the composition can be by orally, buccally, rectally, parenterally, intravenously, intraperitoneally, intradermal, subcutaneously, intramuscularly, trans-dermally, or intratracheally.

An example of the method is wherein theaflavin is administered in an amount equivalent of theaflavin from about 5 mg to about 500 mg per kg of body weight.

As used herein, the term “treating” refers to administering a compound to a subject that has an infectious disease, or has a symptom of or a predisposition toward such a disease, with the purpose to cure, heal, alleviate, relieve, alter, remedy, ameliorate, improve, or affect the above-described disorder, the symptoms of or the predisposition toward it. The term “an effective amount” refers to the amount of the active agent, when used in combination with one or more other active agents, that is required to confer the intended therapeutic effect in the subject.

The above-described pharmaceutical composition can be administered to a subject orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A sterile injectable composition, e.g., a sterile injectable aqueous or is oleaginous suspension, can be formulated according to techniques known in the art using suitable dispersing or wetting agents (such as Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

A composition for oral administration can be any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be io suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. A compound-containing composition can also be administered in the form of suppositories for rectal is administration.

The carrier in the pharmaceutical composition must be “acceptable” in the sense of being compatible with the active ingredient of the formulation (and preferably, capable of stabilizing it) and not deleterious to the subject to be treated. For example, one or more solubilizing agents, which form more soluble complexes with the compounds, or more solubilizing agents, can be utilized as pharmaceutical carriers for delivery of the active compounds. Examples of other carriers include colloidal silicon dioxide, magnesium stearate, sodium lauryl sulfate, and D&C Yellow #10.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are incorporated by reference in their entirety.

### EXAMPLE 1

### Use of Theaflavin to Enhance the Immune System in an Animal Model

To explore therapeutic effects of theaflavin (prepared according to the method described in U.S. Pat. No. 8,282,970) on the immune system, the following experiment was carried out.

Female mice (Kuengming, China) weighing 18-22 g were used. The animals were housed in individual stainless steel cages in an air-conditioned room under a 12:12 hour light:dark cycle. A commercial pellet diet and water were provided throughout the experiment. All procedures were conducted in accordance with the P.R. China legislation under No. 8910M047 on the use and care of laboratory animals.

For each study, forty mice were divided into 4 groups with 10 mice per group (n=10). These four groups were control, theaflavin (low), theaflavin (mid), and theaflavin (high). The latter three were theaflavin-treated groups in which theaflavin (capsule, Xiayie™, prepared according to the method described in U.S. Pat. No. 8,282,970) was fed to mice at a dose of 83 mg, 250 mg, and 500 mg per kg body weight per day for 30 days for the low, mid, and high group, respectively. Water was fed for the control group. For the organ weight study, animals were sacrificed after the 30-day study, followed by collecting spleens and thymus for weight measurement. For the other studies, animals were harvested for further analyses as described below. Statistical differences between groups were calculated using SPSS 11.0 with student t test plus Dunnett method.

Theaflavin treatment showed no effect on mice body weights and weights of immune system related organs, i.e., spleen and thymus, when compared to positive control. See Tables 1 and 2 below.

TABLE 1
NO effect of theaflavin on subject body weight.

| | | | I | | II | | III | | IV |
|---|---|---|---|---|---|---|---|---|---|
| | | n | BW change | N | BW change | N | BW change | n | BW change |
| Group | mg/kg.BW | (mice) | (g) | (mice) | (g) | (mice) | (g) | (mice) | (g) |
| Control | 0 | 10 | 11.80 ± 1.23 | 10 | 12.20 ± 1.03 | 10 | 12.30 ± 0.82 | 10 | 12.30 ± 0.95 |
| Theaflavin (Low) | 167 | 10 | 12.70 ± 0.82 | 10 | 12.00 ± 0.67 | 10 | 11.90 ± 0.88 | 10 | 13.00 ± 0.94 |
| Theaflavin (Mid) | 333 | 10 | 12.80 ± 0.79 | 10 | 11.80 ± 0.63 | 10 | 12.80 ± 0.63 | 10 | 11.90 ± 0.77 |
| Theaflavin (High) | 500 | 10 | 12.50 ± 0.71 | 10 | 12.70 ± 0.92 | 10 | 11.90 ± 0.57 | 10 | 12.00 ± 0.47 |

TABLE 2
NO effect of theaflavin on weights of immune-related organs, i.e., spleen and
thymus.

| | | | Spleen | | | Thymus | | |
|---|---|---|---|---|---|---|---|---|
| | | n | Weight | | | Weight | | |
| Group | mg/kg.BW | (mice) | change (%) | F value | P value | change (%) | F value | P value |
| Control | 0 | 10 | 0.52 ± 0.08 | 0.852 | 0.475 | 0.44 ± 0.05 | 0.066 | 0.978 |
| Theaflavin (Low) | 167 | 10 | 0.47 ± 0.13 | | | 0.42 ± 0.07 | | |
| Theaflavin (Mid) | 333 | 10 | 0.46 ± 0.10 | | | 0.43 ± 0.12 | | |
| Theaflavin (High) | 500 | 10 | 0.45 ± 0.07 | | | 0.42 ± 0.11 | | |

### Mouse Footpad Swelling Assay

Delayed-type hypersensitivity (DTH) is an immune reaction induced by antigen. In the mice footpads at which DTH was elicited, transient swellings which usually peaked at 24-48 hours after the antigen challenge was observed. This assay of measuring footpad swelling of mice caused by a DTH reaction was used to test effects of theaflavin is on immune responses.

The Sheep red blood cell (SRBC) was taken from sheep and washed triple with physiological saline, centrifugated at 1500 g for 10 min, and adjusted to a final concentration of 2%. Each mouse was given a single injection of 0.2 ml of prepared 2% SRBC. On day 4 after the SRBC injection, each mouse was given an intravenous injection (the second injection) of 0.02 ml of prepared 20% SRBC. Mouse footpad thicknesses were measured before and 24 hours after the second injection. Mouse footpad swelling values were calculated from the difference between these two measurements.

The results showed that both theaflavin low and high groups had statistically significant mouse footpad swelling when compared to that of the control. See Table 3 below.

TABLE 3
Effect of theaflavin on DHT in the mouse footpad swelling assay.

| | | n | Footpad | | |
|---|---|---|---|---|---|
| Group | mg/kg.BW | (mice) | swelling (mm) | F value | P value |
| control | 0 | 10 | 0.99 ± 0.48 | 3.505 | |
| Theaflavin (Low) | 167 | 10 | 1.46 ± 0.39 | | 0.041 |
| Theaflavin (Mid) | 333 | 10 | 1.24 ± 0.24 | | 0.396 |
| Theaflavin (High) | 500 | 10 | 1.53 ± 0.49 | | 0.015 |

### Mouse Plasma Hemolysin Assay

Each mouse was given a single injection of 0.2 ml of 2% SRBC from the above-described preparation. On day 5 after the SRBC injection, the plasma was collected by centrifuging the mouse eye blood at 2000 r/min for 10 min, then was stored into a sterile tube. With a micro hemagglutinin-assay plate, the plasma was diluted to a series of concentrations, then 100 ml of 0.5% SRBC suspension was added. The contents were is mixed by vortexing and incubated for 3 h in a 37° C., 5% CO2 incubator, after which the hemagglutination was observed under microscope. The data were presented as antibody integrand.

The results showed that both theaflavin low and high groups had statistically significant results in mouse plasma hemolysin assay when compared to those of the control. See Table 4 below.

TABLE 4
Effect of theaflavin in the mouse plasma hemolysin assay.

| | | n | Antibody | | |
|---|---|---|---|---|---|
| Group | mg/kg.BW | (mice) | integrand | F value | P value |
| control | 0 | 10 | 113.10 ± 21.70 | 3.682 | |
| Theaflavin (Low) | 167 | 10 | 143.80 ± 35.23 | | 0.037 |
| Theaflavin (Mid) | 333 | 10 | 123.20 ± 20.37 | | 0.731 |
| Theaflavin (High) | 500 | 10 | 146.40 ± 26.46 | | 0.022 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present invention, and without departing from the spirit and scope thereof, can make various changes and modifications of the io invention to adapt it to various usages and conditions. Thus, other embodiments are also within the claims.

What is claimed is:

1. A method for enhancing the immune system or for treating an infectious disease, the method comprising administering to a subject in need thereof an effective amount of theaflavins, analogs thereof, or prodrugs thereof.

2. The method according to claim 1, wherein the theaflavins comprises theaflavin, theaflavin-3-gallate, theaflavin-3′-gallate, theaflavin 3,3′-digallate, or a combination thereof.

3. The method according to claim 1, wherein the theaflavins, analogs thereof, or prodrugs thereof are administered to the subject in a dosage equivalent to 5-500 mg of theaflavin per kg of body weight per day.

4. The method according to claim 1, wherein the infectious disease is comprises colds, flu, leprosy, skin infections, acne, a urinary tract infection, or a respiratory infection.

5. The method according to claim 1, wherein the theaflavins, analogs thereof, or prodrugs thereof are in a solid, semi-solid, liquid, or gaseous form.

6. The method according to claim 1, further comprising administering to the subject a second therapeutic agent.

7. A method for enhancing the immune system or improving the immune response sensitivity of the immune system, the method comprising administering to a subject in need thereof a composition comprising a physiologically acceptable carrier and an effective amount of an extract of tea.

8. The method according to claim 7, the extract of tea comprises theaflavin, theaflavin-3-gallate, theaflavin-3′-gallate, theaflavin 3,3′-digallate, or a combination thereof.

9. The method according to claim 8, wherein the extract of tea is administered to the subject in an amount equivalent to 5-500 mg of theaflavin per kg of body weight per day.

10. The method according to claim 8, further comprising catechins, (+)-catechin, (−)-epicatechin, (−)-epigallocatechin, (−)-epicatechin gallate, (−)-epigallocatechin gallate, gallocatechin, catechin gallate, gallocatechin gallate, or a combination thereof.

11. The method according to claim 7, further comprising administering to the is subject a second therapeutic agent.

12. The method according to claim 7, wherein the composition is in a solid, semi-solid, liquid, or gaseous form.

13. A method for treating an infectious disease, the method comprising administering to a subject in need thereof a composition comprising a physiologically acceptable carrier and an effective amount of an extract of tea.

14. The method according to claim 13, the extract of tea comprises theaflavin, theaflavin-3-gallate, theaflavin-3′-gallate, theaflavin 3,3′-digallate, or a combination thereof.

15. The method according to claim 14, wherein the extract of tea is administered to the subject in an amount equivalent to 5-500 mg of theaflavin per kg of body weight per day

16. The method according to claim 14, further comprising catechins, (+)-catechin, (−)-epicatechin, (−)-epigallocatechin, (−)-epicatechin gallate, (−)-epigallocatechin gallate, gallocatechin, catechin gallate, gallocatechin gallate, or a combination thereof.

17. The method according to claim 13, wherein the infectious disease comprises colds, flu, leprosy, skin infections, acne, a urinary tract infection, or a respiratory infection.

18. The method according to claim 13, further comprising administering to the is subject a second therapeutic agent.

19. The method according to claim 13, wherein the composition is in a solid, semi-solid, liquid, or gaseous form.
